(19) United States
(12) Patent Application Publication
(10) Pub. No.: US 2004/0228932 A1
(43) Pub. Date: Nov. 18, 2004

(54) Pharmaceutical excipient

(76) Inventors: Pratibha S. Pilgaonkar, Mumbai (IN); Maharukh T. Rustomjee, Mumbai (IN); Anilkumar S. Gandhi, Mumbai (IN); Vinderjit S. Bhumra, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/439,161

(22) Filed: May 12, 2003

(51) Int. Cl. A61K 35/78; B02C 11/08; B02C 21/00

(52) U.S. Cl. 424/757; 241/23

(57) ABSTRACT

The invention relates to a multifunctional fiber rich fraction (FRF) useful as an excipient for pharmaceutical dosage forms for various routes of administration. This excipient can be used as binder, disintegrant, filler, dispersing agent, coating agent, film forming agent, thickener etc for preparation of variety of dosage forms. This FRF can also be used in a controlled release, targeted release and other specialized delivery systems, as well as in food and cosmetics formulation.

### RELATED APPLICATIONS

None.

### GOVERNMENT INTEREST

None.

### BACKGROUND

The present invention relates to a fraction (here sometimes referred to as the fiber rich fraction or “FRF”) obtained from Trigonella Foenum-graceum seeds, and its use as a pharmaceutical formulation excipient. The present invention entails a novel, solvent-free process for mechanically obtaining this fiber rich fraction from Trigonella Foenum-graceum seed powder. The present invention further discloses a method for manufacturing/obtaining various pharmaceutical dosage forms using this excipient.

Trigonella Foenum-graceum is an herbaceous plant of the leguminous family and is native to Western Asia, from where it has spread widely over Europe, the Mediterranean and rest of Asia. It is one of the oldest cultivated plants and through the ages has found wide application as a food, a food additive and as a traditional medicine of every region in which it has been cultivated. The leaves, and both the ripe and unripe seeds of Trigonella Foenum-graceum are used as vegetables. The seeds also function as a food preservative and are added to pickles, chutneys and other similar food products.

The ripe seed has numerous applications in the traditional medicine system of India. Trigonella Foenum-graceum has been used in treating colic flatulence, dysentery, diarrhea, dyspepsia with loss of appetite, chronic cough, dropsy, enlargement of liver and spleen, rickets, gout and diabetes. The seed is stated to be a tonic. It is also used in post-natal cure and to increase lactation in nursing mothers. Some of the components of Trigonella Foenum-graceum along with the dietary fiber have an important role in the treatment and management of several disorders such as obesity, coronary heart disease, diabetes, piles, fissures, chronic constipation & diverticulitis. The saponins are also reported to contain active components that are anti-carcinogenic, anti-microbial and/or anti-oxidant. However, the dose required for treatment of these diseases is very high and typically ranges from 10-12 grams per day.

Organic solvent-based methods for isolating various components of the Trigonella Foenum-graecum plant are known in the art. For example, Peter CHANG, U.S. Pat. No. 5,997,877, discloses a process for using various organic solvents for the fractionation of Trigonella Foenum-graecum seeds and the extraction of the various fractions thereof. The process described gives a high yield rate, and provides a number of high-quality fractions of the Trigonella Foenum-graecum seed including a soluble dietary fiber fraction, de-flavored Trigonella Foenum-graceum seed, high-protein Trigonella Foenum-graceum meal, and dioscin and other saponins, along with the Trigonella Foenum-graceum oleoresins which have conventional commercial use. Similarly, BOURRET, PCT application WO/0174371A1 disclosed a method of using organic solvents to obtain Trigonella Foenum-graceum mucilage in the form of flour with grain size distribution less than 100 μm, consisting mannose, galactose, glucose, arabinose, xylose, rhamnose, D-galacturonic acid, galactomannans, and proteins. Similarly, PCT application WO 0128673A1 describes the manufacture or isolation of galactomannans using various organic solvents, and the use of such galactomannans.

Similarly known in the art are various uses of such organic solvent-extracted fractions of Trigonella Foenum-graceum seed. For example, BLANK et al., U.S. Pat. No. 6,013,289, discloses a flavorant composition prepared by hydrolyzing Trigonella Foenum-graecum seed material with enzymes. Similarly, SHUKLA, U.S. Pat. No. 6,372,220, describes coating perishable substances such as edible substances or foods by extending their freshness, shelf life or suitability for consumption by living organisms. Specifically, the material is impregnated with an extract of Trigonella Foenum-graecum.

In contrast to these references, we have found a way to isolate a fiber rich fraction (FRF) from Trigonella Foenum-graceum seed, without the use of organic solvents at all. We do this by using an elegantly simple mechanical procedure, which leaves neither organic residues nor toxic byproducts in the FRF. Because the FRF is made without the use of organic solvents, it is extraordinarily pure and safe; this purity and lack of organic solvent contamination makes FRF exceptionally valuable as an excipient in pharmaceutical manufacturing. FRF can be used as release retarding polymer, a binder, a suspending agent, coating agent, a gelling agent, a film forming agent, a diluent and as a carrier for drug. Before the discovery of the present invention, Trigonella Foenum-graecum galactomannan was considered both more water-soluble and a less efficient thickening agent than the well-known gums. However, it was unexpectedly found that the fiber rich fraction has comparable viscosities to the polymers reported in the literature.

### ADVANTAGES OF THE INVENTION

The fiber rich fraction described in the present invention offers a number of advantages to a pharmaceutical formulation scientist. It is a novel multifunctional excipient, which can be used for a variety of applications in pharmaceuticals. This novel excipient can be used as a release retarding polymer, a binder, a suspending agent, a gelling agent, coating agent, a film forming agent, a diluent and as a carrier for drug. It acts as a good release retarding polymer for drugs with varying solubilities, particularly for highly soluble drugs, which are difficult to formulate in controlled release formulation. It also acts as a good suspending agent. It can be used in all liquid, semisolid and solid dosage forms. It can be formulated for oral, nasal, ocular, urethral, buccal, transmucosal, vaginal, topical or rectal delivery. It presents no toxicity risks as it is “GRAS” (“Generally Recognized as Safe” by the United States Food & Drug Administration) listed. It can be adopted for large-scale production.

Various features of novelty that characterize the present invention are pointed out with particularity in the claims annexed to and forming a part of this disclosure. For a better understanding of the invention, its advantages and objects, reference is made to the accompanying drawing descriptive matter in which a preferred embodiment of the invention is illustrated.

### SUMMARY OF THE INVENTION

Many prior art references disclose method of isolating various components from Trigonella foenum-graceum seeds, and various uses of these components. The art fails to teach the use of FRF as a pharmaceutical excipient.

An object of the present invention is to establish FRF as a multifunctional pharmaceutical excipient, and its use as a release retarding polymer, a binder, a suspending agent, a gelling agent, a film forming agent, a diluent and as a carrier for drug. The present invention includes methods of obtaining FRF from Trigonella foenum-graceum seeds by mechanical or by solvent treatment. The present invention also includes pharmaceutical compositions containing the fiber rich fraction.

### BRIEF DESCRIPTION OF THE FIGURES

FIG. 1: Release profiles of metformin hydrochloride from METHOCEL® brand cellulose and FRF matrix, showing the comparative dissolution of metformin hydrochloride tablets prepared using FRF, and prepared using METHOCEL® brand cellulose.

### DETAILED DESCRIPTION

A drug is defined as an agent intended for use in the diagnosis, mitigation, treatment, cure or prevention of disease in humans or in other animals. One of the most astounding qualities of drugs is the diversity of their actions and effects on the body. Drug substances are seldom administered alone, but rather as part of a formulation in combination with one or more non-medical agents that served varied and specialized pharmaceutical function, which are termed as pharmaceutical ingredients or excipients. These pharmaceutical excipients solubilize, suspend, thicken, dilute, emulsify, stabilize, preserve, color, flavor and fashion medicinal agents into efficacious and appealing dosage forms.

We use the term “excipient” here to mean a non-pharmaceutically active additives used in the manufacture of a pharmaceutical composition, which additive allows the pharmaceutically active ingredient or medicament to be manufactured into a pharmaceutical formulation or a galenic formulation which provides the necessary bioavailability of the medicament to the patient upon the administration of the pharmaceutical composition. The bioavailability of a given dosage form is dependent on process variables and the interrelationship between the various excipients and the active ingredient.

Over the past four decades, excipient suppliers have developed novel mixtures and new physical forms of excipients that are tailored to the needs of newer pharmaceutical delivery platforms (transdermal, pulmonary, oral modified release). Most excipient manufacturers, however, concentrate on modifying existing pharmaceutical excipients, as opposed to developing entirely new ones. This is because Food & Drug Administration regulatory restrictions have made innovation rather expensive and difficult.

However, there remains a need for excipients that serve a variety of purposes in the finished pharmaceutical formulation, that serve as a specialized excipient for novel drug delivery systems, and yet are inexpensive.

The present invention describes use of the fiber rich fraction as a novel excipient for a number of pharmaceutical applications. This novel excipient can be used in a wide variety of solid, semisolid and liquid preparations meant for different routes of administration.

Drugs are most frequently taken by oral administration. Although a few drugs taken orally are intended to be dissolved in mouth, the vast majority of drugs taken orally are swallowed. Of these, the most are taken for the systemic drug effects that result after absorption from the various surfaces along the gastrointestinal tract. Compared with alternate route, the oral route is considered the most natural, uncomplicated, convenient and safe means of administering drugs. Drugs administered by the oral route in a variety of pharmaceutical forms. The most popular are tablets, capsules, suspensions and various pharmaceutical solutions.

When medications are to be administered orally to adults, capsules and tablets usually are preferred because they are conveniently carried, readily identified and easily taken. Moreover, these dosage forms allow a good accuracy of dosage of the active component of the medicinal formulation. Furthermore, as no liquids are generally involved in the process for preparing these medicinal formulations, handling and packaging are a lot easier. Last but not least, conservation and stability of these preparations are generally better than those of other formulations.

The International Pharmaceutical Excipients Council (IPEC) spells out thirteen general categories of excipients for pharmaceutical solid dosage forms. These thirteen categories are based on the excipients' function in the formulation. The functions are: binders, disintegrants, fillers, lubricants, glidants, compression aids, colors, sweeteners, preservatives, suspending /dispersing agents, film formers/coatings, flavors, and printing inks.

When the excipient of the present invention is used for manufacture of solid dosage forms, it can act as, inter alia, a diluent, binder, disintegrant and coating agent.

Binders are used to hold together the structure of the dosage forms. They have the property to bind together all the other ingredients after sufficient compression forces have been applied and they provide the physical, structural integrity of the tablets. Tablets can be manufactured using three main processes: wet granulation, dry granulation and direct compression. In wet granulation, components are typically mixed and granulated using a wet binder, the wet granulates are then sieved, dried and eventually ground prior to compressing the tablets. In dry granulation, powdered components are typically mixed prior to being compacted, also called pre-compression, to yield hard slugs which are then ground and sieved before the addition of other ingredients and final compression.

FRF can act both as dry as well as wet binder. It can be used at a concentration ranging from 0.5 to 20% or more by weight of the total formulation. As a wet binder, FRF may be dissolved in water and the resulting solution can be employed to bind the remaining part of the formulation, such as a powder mass of active pharmaceutical ingredient. Similarly, FRF may be used as a dry binder, being dry mixed with the active pharmaceutical ingredient and the admixture later granulated using dematerialized water. FRF may also be employed as a dry binder in case of direct compression. Tablets with good hardness value, acceptable disintegration time and friability may thus be made using FRF.

When the tablet is used as a pharmaceutical product, in addition to the above-mentioned requirement of structural strength, the disintegration time of the tablet must be short enough for the tablet to express its pharmacological effect after the tablet is orally taken. Generally, after orally taken, a tablet is disintegrated in digestive tracts, and then the active pharmaceutical ingredient is dissolved in a digestive liquid. Therefore, when a tablet disintegrates in digestive tracts immediately after it is orally taken, a rapid expression of the pharmacological effect of the active ingredient contained in the tablet can be obtained.

Disintegrants may be added to pharmaceutical formulations in order to help the tablets disintegrate when they are placed in a liquid environment and so release the active ingredient. The novel excipient FRF acts as a disintegrant. It can be generally used at a concentration ranging from 0.5 to 20% or more of the finished dosage form weight. The disintegration properties are based upon the ability of the FRF to swell in the presence of a fluid such as water or gastric juice. This swelling disrupts the physical continuity of the tablet structure, leading to its physical disintegration.

Another important class of oral dosage forms includes syrups, suspensions and emulsions. In these systems the pharmaceutical active ingredient may be dissolved in an aqueous or non-aqueous solvent or combination, by suspending the pharmaceutical active ingredient (if it is insoluble) in an appropriate medium, or by incorporating the pharmaceutical active ingredient into one of the two phases of an oil and water system. These dosage forms are useful for a number of reasons. They can be formulated for various routes of administration: oral use, introduction into body cavities or external application. The dose can be easily adjusted and more importantly it can be readily administered to children or aged people or people who cannot swallow tablets or capsules. One of the important aspects of these dosage forms is the viscosity, which is required to prevent sedimentation rate of the solids in suspension and to achieve desired stability in case of emulsion and to arrive at a consistency suitable for administration in case of solutions. Thickening agents or agents imparting viscosity are therefore an important additive in these formulation.

The FRF can be employed as a thickening agent for pharmaceutical active ingredient solutions, suspension or emulsions. FRF can be used at a concentration level from 0.5 to 20% or more w/v. The actual concentration of FRF can be selected based on the desired consistency.

Another important aspect of disperse systems is the agent that stabilizes the system. This can be the suspending agent or the emulsifying agent. FRF can be used as a suspending agent or as an emulsifying agent at a concentration ranging from 0.5 to 20% or more w/v.

Ointments, creams and gels are semisolid dosage forms intended for topical application. They may be applied to the skin, placed on the surface of the eye, or used nasally, vaginally or rectally. The majority of these preparations are used for the effects of the therapeutic agents they contain. Those, which are non-medicated, are used for their physical effects as protectants or lubricants. Some topical applications, most notably transdermal drug delivery systems are designed for the systemic absorption of drug substances in therapeutic quantities. Important excipients of these systems are agents that increase the viscosity and give the semisolid nature of these preparations. A number of bases such as hydrocarbon bases, absorption bases water removable bases etc are reported in literature for ointments. For creams and gels a number of polymers are listed in the literature.

The FRF can be employed as a thickening agent, base or gelling agent for these semisolid formulations. It can be used at a concentration level from 0.5-50% w/v. The concentration can be selected based on the desired consistency, appearance and the desired physical and chemical properties of the final product.

Tablets are coated for a number of reasons, including to: protect the medicinal agent against destructive exposure to air and/or humidity, mask the taste of the drug, provide special characteristics of drug release and to provide aesthetic or distinction to the product. There are four major techniques employed for coatings to the pharmaceutical dosage forms: sugar coating, film coating, micro-encapsulation and compression coating. Most widely employed technique today is film coating. A number of polymers such as cellulosics, methacrylate copolymers, polyvinyl alcohol etc are used for coating purposes as solutions in water or organic solvents or as aqueous dispersions.

The FRF can be employed as a coating agent for coating of various dosage forms. It can be used at a concentration level from about 0.5 to 20% or more w/v. The concentration can be selected based on the desired consistency, appearance and the desired physical and chemical properties of the final product. Commonly employed plasticizers such as polyethylene glycol can be also be used along with opacifiers and colorants and other excipients.

The goal of any drug delivery system is to provide a therapeutic amount of drug to the proper site in the body to achieve promptly and then maintain the desired drug concentration. That is, the drug delivery system should deliver drug at a rate dictated by the need of the body over a specified period of treatment. This idealized objective points to the two aspects most important to drug delivery namely special placement and temporal delivery of a drug. Spatial placement relates to targeting a drug to a specific organ or tissue, while temporal delivery refers to controlling the rate of drug delivery to the targeted tissue. Temporal delivery also therefore refers to the modified release systems. The advantages of modified release products are well known in the pharmaceutical field and include the ability to maintain a desired blood level of a medicament over a comparatively longer period of time while increasing patient compliance by reducing the number of administrations necessary to achieve the same.

For controlled release systems, the oral route of administration has received the most attention. The commonly used approaches to control the release of a drug include diffusion controlled systems, dissolution controlled systems, osmotic systems, ion exchange, prodrugs, swelling and expanding systems etc. Among all these approaches matrix approach is commonly used, as it is easy to formulate dosage forms at large scale at relatively low costs. This approach involves drug release via diffusion and dissolution. In a such systems the rate of dissolution of drug is reduced by, for example, embedding the drug in a polymeric matrix or surrounding it with a polymeric barrier membrane through which drug must diffuse to be released for absorption.

A number of polymers are reported for this purpose which includes cellulose polymers and their derivatives including, but not limited to, hydroxymethyl cellulose, hydroxyethyl cellulose, hydroxypropyl cellulose, hydroxypropylmethyl cellulose, carboxymethylcellulose, microcrystalline cellulose, ethyl cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, maltodextrins, poly (2-ethyl-2-oxazoline), poly(ethyleneimine), polyurethane hydrogels, crosslinked polyacrylic acids and their derivatives etc.

The FRF can be employed as a release retarding polymer for controlling the release of drug from various dosage forms. As a release retarding polymer, FRF can be used at a concentration level from about 5 to about 95% w/v. The concentration can be selected based on the desired release profile, and the nature and dose of the active pharmaceutical ingredient. This FRF can be used alone or in combination with above mentioned polymers. FRF can be effectively used for drugs with varying solubilities such as very soluble (1 part in less than 1 part of water), freely soluble (1 part in 1 to 10 parts of water), soluble from (1 part in 10 to 30 parts of water), sparingly soluble (1 part in 30 to 100 parts of water), slightly soluble (1 part in 100 to 1000 parts of water), very slightly soluble (1 part in 1000 to 10,000 parts of water), and practically insoluble (1 part in more than 10,000 parts of water). FRF can thus be used for controlled delivery of both lipophilic and hydrophilic drugs. Also, it can be conveniently employed for large, medium and low dose drugs, alone or in combination. FRF can be incorporated in the various dosage forms that can be used for controlling the drug release such as capsules, tablets, micro granules, pellets, coated systems, etc.

Films or Patches

These delivery systems are mainly developed for transdermal application as well as application to the mucosal tissues such as oral mucosal, eye, vagina etc. When use for buccal or oral mucosal delivery system, they avoid some of the drawbacks of tablets, and provide rapid onset of action, a large surface for contact with mucosal and more importantly it does not give a feeling of extraneous matter in the cavity as is the case with tablets. These drug loaded adhesive films can be designed either for unidirectional release into the oral mucosal of for bidirectional release.

Dosage forms employed for topical drug delivery such as creams, gels, emulsions and lotions include, e.g. the lack of precision of the application of dosage form and lack of control over precise doses to the target site. Also these dosage forms will not be useful for transdermal delivery of drugs across skin for a longer duration. Polymeric films have therefore been developed to overcome these problems. In these systems the drug is distributed within a thin, hydrophilic/hydrophobic adhesive film. For several years, transdermal drug delivery systems have been employed to effectively introduce certain drugs into the bloodstream through unbroken skin. Aside from comfort and convenience, transdermal systems avoid the gastro-intestinal tract, the delivery rate control problems and potential toxicity concerns associated with traditional administration techniques, such as oral, intramuscular or intravenous delivery. For example, such systems have proven particularly effective in the delivery of melatonin and other natural hormones to the body, since transdermal delivery mimics the body's own system of secretion. Transdermal delivery has traditionally involved the transport of a drug or drugs across the stratum corneum, the layer of the skin responsible for preventing water loss and the transport of substances through the skin, and into the bloodstream. Transdermal devices known in the art include reservoir type devices including membranes, pressure-sensitive adhesive matrices and skin patches.

The FRF can be employed as a structural component in a film or patch formulation. As such, FRF can be used at a concentration level from 0.5 to about 50% or more w/v. The concentration can be selected based on the desired consistency, appearance and the desired physical and chemical properties of the final product. Mechanical properties of the film are extremely important. These include tensile strength, modulus of elasticity, percent elongation at break, folding endurance water uptake, flatness etc

The FRF can be incorporated in the following dosage forms: a capsule, a tablet, an ovule, a suppository, an insert, a wafer, a chewable tablet, a buccal tablet, a sub-lingual tablet, a quick-dissolve tablet, an effervescent tablet, a granule, a pellet, a bead, a pill, a sachet, a sprinkle, a film, an ointment, a cream, a gel, a dry syrup, a reconstitutable solid, a suspension, an emulsion, a lozenge, a troche, an implant, a powder, a triturate, a platelet, or a strip. These pharmaceutical compositions can be formulated for immediate release, pulsatile release, controlled release, extended release, modified release, delayed release, targeted release, or targeted delayed release. Also the compositions can be formulated for oral, nasal, ocular, urethral, buccal, transmucosal, vaginal, topical or rectal delivery. For development of these dosage forms this novel excipient can be combined with other excipients such as water soluble polymer, water insoluble polymers, hydrophobic materials, hydrophilic materials, waxes, disintegrants, superdisintegrants, diluents, binders, etc.

Making FRF:

FRF can be obtained from Trigonella foenum-graceum seed. This seed comprises of a central hard, yellow embryo surrounded by a corneous and comparatively large layer of white semi-transparent endosperm. This endosperm contains galactomannan gum. The endosperin is surrounded by a tenacious dark brown husk. In the process of making FRF, we separate the endosperm and husk portion (the fiber rich fraction or “FRF”) from the embryo. FRF is the endosperm and the husk of the seed.

The seeds were selected based on the dimension and swelling factor. Seeds having dimensions between those given here were selected. Seeds having length between 3.0-6.0 mm, preferably 3.5-4.5 mm were selected. Breadth of the seed ranged from 1.5-4.0 mm, preferably 2.0-3.5 mm. The swelling factor ranged from 0.5 to 50.0, preferably 3.0 to 35.0.

Thus selected, seeds were treated mechanically to isolate the somewhat translucent FRF. The mechanical treatment process entails milling the selected Trigonella foenum-graceum seeds in a multimill with hammer forward at fast speed. This results in physical separation of FRF from the yellow embryo, to make a mixture of the two components physically together, but no longer physically attached to each other. Various different types of mills available such as fritz mill, hammer mill, colloid mill, roller mill, multimill etc can be employed for the isolation.

Separating fiber rich fraction from the yellow embryo may be carried out by sieving through a sieve. For example, one may use a 16 # sieve; the fraction below 16 # (i.e., the yellow embryo-rich fraction) is discarded. We do not, however, restrict ourselves to a particular sieve size. This is because the yellow embryo gets powdered relatively easily, whereas the husk and endosperm (the FRF) is fibrous and thus difficult to powder, and remains as course particles. As such, FRF can be separated from the powdered embryo fraction by sieving using a variety of sieve sizes. Sieves from 8 # to 40 #, or even 100 # could be employed; the sieve size need simply be adequate to separate the yellow endosperm from the FRF, so the sieve size will depend on the size of the yellow embryo powder particle size and of the FRF, each affected by, for example, the intensity of milling.

Alternatively, the mechanical separation could be achieved by a mechanical process of roasting, differential attrition, sieving and polishing which can also be performed on the large scale.

The retained fraction is again milled and sieved. (This milling process is repeated in order to minimize the yellow embryo portion of the seed which was monitored visually.)

The resulting fraction is FRF. If required fiber rich fraction thus obtained is further milled using a roller mill or any other suitable mill to the desired particle size.

Some of the advantages of the developed process are: (1) The developed process is solvent free, which itself is environmentally friendly, has no carryover of solvent residues in the final product, is cost effective, and no solvent recovery systems required; (2) the process is relatively simple and free of complicated steps; (3) the process can be carried out using standard equipment available with pharmaceutical industry and no special equipment is required; and (4) the FRF thus obtained is free from bitter principles.

The FRF can also be obtained using debitterised Trigonella Foenum-graceum or using commercially available dietary fibers such as FENULIFE® brand dietary fiber, a fiber derived from a variety of the Trigonella Foenum-graceum plant selected for its high soluble fiber content and lack of odor. The preparation of FENULIFE®) brand dietary fiber is disclosed in U.S. Pat. No. 5,997,877; FENULIFE® brand dietary fiber is commercially available from Acatris, Inc., Minneapolis, Minn.

Alternatively, the selected seeds can be treated with solvents in order to isolate the desired component of the seed. In yet another embodiment the isolation could be carried out using a combination of milling and solvent extraction. Various different types of mills available such as fritz mill, hammer mill, colloid mill, roller mill, multimill etc can be. employed for the isolation. Solvent employed for extraction could be any aqueous or non-aqueous solvent. Examples of solvents include water, ethanol, methanol, methylene chloride, chloroform, hexane, ethylene chloride, and the like. The process employed for the isolation may involve soxhlet extraction, maceration, percolation or any other methods of extracting plant constituents. The fraction used as novel excipient is a fiber rich fraction.

### EXAMPLE 1

### Use of the FRF as a Controlled Release Vehicle for a Highly Water Soluble Drug—Metformin Hydrochloride

| Ingredients | Lot A (mg/unit) | Lot B (mg/unit) |
|---|---|---|
| Metformin HCl | 500 | 500 |
| METHOCEL ® | — | 360 |
| Fiber rich fraction | 360 | — |
| Sodium Carboxymethyl cellulose | 100 | 100 |
| Magnesium stearate | 5 | 5 |

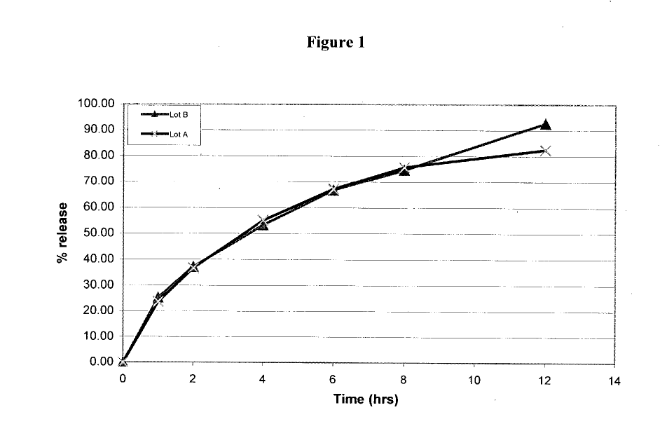

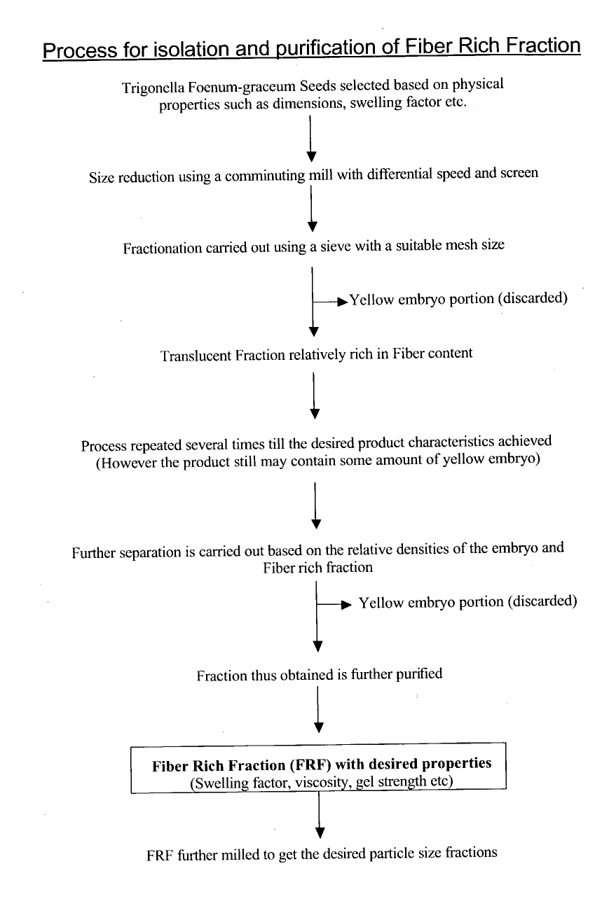

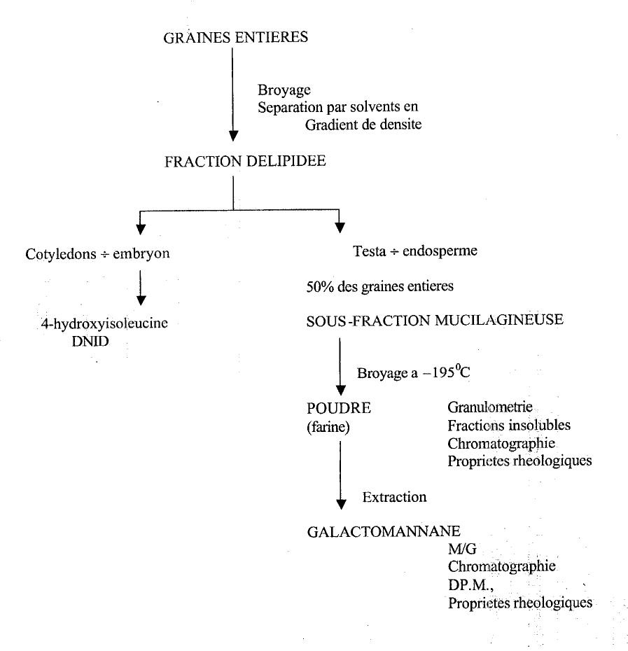

Metformin was granulated and the granules divided into two lots. Lot A granules were lubricated with Fiber rich fraction and sodium carboxymethyl cellulose and compressed into tablets after lubrication with magnesium stearate. Lot B granules were lubricated with METHOCEL® and sodium carboxymethyl cellulose and compressed tablets after lubrication with magnesium stearate. METHOCEL® brand cellulose ethers, commercially available from the Dow Chemical Company, Midland, Mich., are water-soluble methylcellulose and hydroxypropyl methylcellulose polymers that bind, retain water, thicken, form films, lubricate, and add unique physical properties to carious applications. Dissolution was carried out using 900 ml 6.8 phosphate buffer in USP apparatus 1 Basket). The following table, shown graphically at FIG. 1, indicates that both the formulations are comparable (F2 value=67.74).

Average % release

| Time intervals | Example 1 | Example 1 |
|---|---|---|
| (hrs) | Lot A | Lot B |
| 1 | 25.24 | 23.64 |
| 2 | 37.18 | 36.66 |
| 4 | 53.32 | 55.12 |
| 6 | 66.82 | 67.23 |
| 8 | 74.64 | 75.74 |
| 12 | 92.7 | 82.51 |

This graph shows that the two curves are almost super-imposable and the following inferences can be drawn from these results:

1. The Fiber rich fraction can be used as a sustained release excipient for highly water-soluble drug such as metformin hydrochloride.

2. The Fiber rich fraction can act as a controlled release vehicle at a concentration equivalent to METHOCEL® concentrations.

### EXAMPLE 2

### Use of the FRF as a Disintegrating Agent

The fiber rich fraction was incorporated in citalopram tablets and compared with tablets without this excipient.

| | Lot A | Lot B |
|---|---|---|
| Excipients | (mg/tablet) | (mg/tablet) |
| Citalopram hydrobromide | 49.96 | 49.96 |
| Microcrystalline cellulose | 120.00 | 112.00 |
| Lactose | 184.04 | 184.04 |
| Pregelatinised starch | 30.00 | 30.00 |
| Fiber rich fraction | — | 8.00 |
| Magnesium stearate | 4.00 | 4.00 |
| Tablet weight (mg) | 388.00 | 388.00 |
| Hardness (Kg/cm²) | 7-8 | 7-8 |
| Disintegration time (min.) | >10 min | 7-8 |

Citalopram hydrobromide tablets were prepared using the Fiber Rich Fraction as a disintegrant. A control was also prepared without the novel excipient and disintegration time was recorded using Electrolab disintegration test apparatus. The formulation containing the Fiber rich fraction exhibited a disintegration time of 7-8 min whereas for tablets without this excipient exhibited a disintegration time of greater than 10 min. which suggest that the Fiber rich fraction can be used as a disintegrating agent for tablets and capsules.

### EXAMPLE 3

### Use of the FRF as a Binder

Binding properties of the Fiber rich fraction was studied in this experiment. In one embodiment (Lot A) the novel pharmaceutical excipient FRF was dry mixed with the drug and other formulation excipients and the blend was granulated using water. In another embodiment (Lot B) the FRF was dispersed in water and this solution was then used for granulation of the blend.

| | Lot A | Lot B |
|---|---|---|
| Ingredients | (mg/tablet) | (mg/tablet) |
| Simvastatin | 40.00 | 40.00 |
| Microcrystalline cellulose | 250.96 | 260.96 |
| Lactose | 75.00 | 75.00 |
| Fiber rich fraction | 20.00 | 10.00 |
| Magnesium stearate | 2.00 | 2.00 |
| Butyrate hydroxy anisole | 0.04 | 0.04 |
| Tablet weight (mg) | 388.00 | 388.00 |
| % fines (−100 #) | 8.69 | 20 |
| % compressibility | 16.29 | 18.74 |
| Hardness (Kg/cm²) | 6-7 | 7-8 |
| Disintegration time (min.) | 5-6 | 3-4 |

Thus it can be seen from the above table that the novel pharmaceutical excipient FRF has good binding property. This is evident from the hardness achieved, % fines obtained in the granules and the compressibility of the blend.

### EXAMPLE 4

### Use of the Novel Excipient FRF as a Thickening Agent

Owing to a high swelling properties of the FRF, it can be used effectively in the preparation of gels, ointments creams etc. the following example demonstrates the use of the Fiber rich fraction as a thickening agent in diclofenac gel.

| Ingredients | grams |
|---|---|
| Diclofenac diethylamine | 0.58 |
| Propylene glycol | 2.5 |
| Fiber rich fraction | 3.75 |
| Distilled water | 43.17 |

Diclofenac diethylamine was dissolved in propylene glycol and was dispersed in gel prepared by hydrating the Fiber rich fraction. The gel thus obtained had good consistency, good extrudability and spreadability. Thus the novel excipient Fiber rich fraction can be used as a thickening or gelling agent.

### EXAMPLE 5

### Use of the FRF as a Film-Forming Agent

A part of swelling properties that the novel excipient FRF has, its ability to form free as well as medicated film was evaluated.

| Ingredients | Grams |
|---|---|
| Diclofenac diethylamine | 1.16 |
| Propylene glycol | 1.5 |
| Fiber rich fraction | 3.0 |
| Distilled water (q.s.) | 100 |

The FRF was dispersed in distilled water sifted through 80 #. To this sifted solution diclofenac diethylamine previously dispersed in propylene glycol was added. The resulting solution was poured in siliconised Petri dishes and dried in a tray drier at 60° C. for about 8-12 hours. The resulting film had desired elasticity, flatness, and tensile strength. Thus the novel excipient can be used for the preparation of films that might be employed for transdermal or buccal or any other application.

### EXAMPLE 6

### Use of FRF as a Coating Agent

As this FRF has good film forming properties, it should be readily used as a coating agent for film coating of tablets. The following formula was used for the preparation of film coating solution.

| Ingredients | Grams/batch |
|---|---|
| Fiber rich fraction | 8.00 |
| Propylene glycol | 1.50 |
| Talc | 18.00 |
| Titanium dioxide | 18.00 |
| Quinoline yellow lake | 1.5 |
| Distilled water | 353.00 |

The FRF dispersed in distilled water and propylene glycol. Talc and titanium dioxide was added to this dispersion. Finally quinoline yellow lake was added and the solution was passed through 80 # sieve. The resulting solution was employed for coating of a placebo tablets. Samples were removed at different levels of coating. Coating was completely smoothly and the defects such as logo bridging, orange peel effect, etc was not observed. Formation of a uniform coat suggested the application of novel excipient derived from Trigonella foenum-graceum seed powder as a coating agent.

### EXAMPLE 7

### Use of the FRF as a Suspending Agent

| Ingredients | Qty/100 ml |
|---|---|
| Sugar syrup | 50.20 |
| Calcium carbonate | 12.70 |
| Sorbitol | 10.00 |
| Glycerin | 10.00 |
| Fiber rich fraction (FRF) | 3.00 |
| Erythrosine solution (2%) | 0.10 |
| Raspberry flavor | 0.10 |
| D.M water | q.s |

The FRF was used as a suspending agent at a concentration of 3% in a high dose calcium suspension. Viscosity of the solution was determined using Brookfield viscometer and was found to be about 2000 cps at 25° C. The sedimentation rate of the formulation was determined over a period of 30 days under ambient conditions and almost no sedimentation was observed. Our FRF pharmaceutical excipient can be used effectively as a suspending agent.

### EXAMPLE 8

### Use of the FRF in Shampoo Containing Herbal Extracts

| Ingredients | Quantity (% w/w) |
|---|---|
| SSC-200 base | 50.00 |
| Herbal extract | 2.00 |
| Formalin | 0.10 |
| Silicon oil | 2.00 |
| Fiber Rich Fraction | 5.00 |
| pH adjusting agent (dil. | q.s. |
| HCl or NaOH) | |
| Color | 0.25 |
| Perfume | 1.00 |
| Purified water | q.s. |

Procedure:

The aqueous dispersion of FRF was added into the SSC-200 base. To this mixture herbal extract was incorporated under constant stirring. Then formalin and perfume were added to the mixture, and the pH was adjusted to 6-7. Finally silicon oil emulsion was added and stirred to get a homogenous shampoo. The required amount of color was added in the form of aqueous solution and the weight is adjusted with water. The shampoo thus obtained was evaluated for viscosity, wetability, pH and other parameters. The formulated shampoo using FRF exhibited the desired properties and it was stable over a period of time.

Summary:

It will become apparent to one of skill in the art that our invention may be modified to create products which vary from the examples discussed herein, but still fall within the spirit and scope of our invention as claimed. For example, the ratios of FRF to active pharmaceutical ingredient may be varied, as may the nature and number of other excipients used along with the FRF. Similarly, other methods of mechanical separation of the FRF from the embryo work equivalently to the specific examples taught here. Thus, while we discuss several preferred embodiments in detail, we intend the legal coverage of our patent to be defined not by the specific examples discussed, but by the legal claims appended here.

We claim:

1. A product-by-process pharmaceutical excipient comprising the fiber rich fraction of Trigonella foenum-graceum seeds, said pharmaceutical excipient made by mechanically separating the fiber rich fraction of said seeds from the embryo of said seeds.

2. The product-by-process of claim 1, said mechanical separation comprising:
a. milling said seeds; and
b. sieving said milled seeds in a sieve sized to separate the embryo of said seeds from the fiber rich fraction of said seeds.

3. The product-by-process of claim 2, said sieve having a mesh size of from about 8 # to about 100 #.

4. The product-by-process of claim 1, said mechanical separation comprising the following steps in the following order:
a. roasting said seeds,
b. differential attrition of said seeds,
c. sieving said seeds in a sieve sized to separate the embryo of said seeds from the fiber rich fraction of said seeds and
d. polishing said fiber rich fraction.

5. A pharmaceutical composition of matter comprising:
a. an active pharmaceutical ingredient, and
b. the excipient of claim 1.

6. The composition of matter of claim 5, said excipient present in an amount effective to retard the in vivo release of said active pharmaceutical ingredient.

7. The composition of matter of claim 6, said amount effective to retard the in vivo release of said active pharmaceutical ingredient being from about 5% to about 95% of the total weight of said composition of matter.

8. The composition of matter of claim 5, said excipient present in an amount effective to aid the in vivo disintegration of said composition of matter.

9. The composition of matter of claim 8, said amount effective to aid the in vivo disintegration of said composition of matter being from about 0.5% to about 20% of the total weight of said composition of matter.

10. The composition of matter of claim 5, said excipient present in an amount effective as a binder for said active pharmaceutical ingredient.

11. The composition of matter of claim 10, said amount effective as a binder ranging from about 0.5% to about 20% of the total weight of said composition of matter.

12. The composition of matter of claim 5, said excipient present in an amount effective as a suspending agent.

13. The composition of matter of claim 5, said excipient present in an amount effective as a gelling agent.

14. The composition of matter of claim 5, said excipient present in an amount effective as a film-forming agent.

15. The composition of matter of claim 5 in a physical configuration selected from the group consisting of: a capsule; a tablet; an ovule; a suppository; an insert; a wafer; a chewable tablet; a buccal tablet; a sub-lingual tablet; a quick-dissolve tablet; an effervescent tablet; a granule; a pellet; a bead; a pill; a sachet; a sprinkle; a film; an ointment; a cream; a gel; a lotion; a dry syrup; a reconstitutable solid; a solution; a suspension; an emulsion; a lozenge; a troche; an implant; a powder; a triturate; a platelet; and a strip.

16. The composition of matter of claim 5 further comprising a compound selected from the group consisting of: a water soluble polymer; a water insoluble polymer; a wax; a diluent; and a super-disintegrant.

17. The composition of matter of claim 15 formulated for a delivery route selected from the group of: oral delivery; nasal delivery; ocular delivery; urethral delivery; buccal delivery; transmucosal delivery; vaginal delivery; topical delivery and rectal delivery.

18. The composition of matter of claim 7, said excipient present in an amount to effect an active pharmaceutical ingredient release profile selected from the group consisting of: immediate release; pulsatile release; controlled release; extended release; delayed release; modified release; targeted release; and targeted delayed release.
